(19) United States
(12) Patent Application Publication
(10) Pub. No.: US 2018/0120320 A1
(43) Pub. Date: May 3, 2018

(54) NOVEL METHOD FOR ANALYZING CIRCULATING TUMOR CELLS OF A PATIENT FOR THE PRESENCE OF METASTASIS-INITIATING CELLS

(71) Applicant: HI-STEM Heidelberg Institute for Stem Cell Technology and Experimental Medicine gGmbH, Heidelberg (DE)

(72) Inventors: Andreas Trumpp, Heidelberg (DE); Irène Baccelli, Heidelberg (DE)

(21) Appl. No.: 15/796,647

(22) Filed: Oct. 27, 2017

Related U.S. Application Data
(62) Division of application No. 13/415,481, filed on Mar. 8, 2012.

(51) Int. Cl. G01N 33/574

(52) U.S. Cl. CPC G01N 33/57415; G01N 33/57492; G01N 2333/70585; G01N 2333/70596; G01N 33/57488

(57) ABSTRACT

This invention relates to a novel method for analyzing circulating tumor cells of a patient for the presence of metastasis-initiating cells. The method comprises the step of detecting cells exhibiting the simultaneous presence of c-MET, CD44 and CD47. The invention further relates to novel kits, novel methods for treating patients, and novel bifunctional analytes.

### FIELD OF THE INVENTION

This invention relates to a novel method for analyzing circulating tumor cells of a patient for the presence of metastasis-initiating cells. The method comprises the step of detecting cells exhibiting the simultaneous presence of c-MET, CD44 and CD47. The invention further relates to novel kits, novel methods for treating patients, and novel bifunctional analytes.

### BACKGROUND OF THE INVENTION

Metastatic spread is a complex process initiated by the dissemination, seeding and engraftment of malignant cells in sites distant to the primary tumor (Kim et al., Tumor self-seeding by circulating cancer cells. Cell 139 (7), 1315-1326 (2009); Nguyen et al., Metastasis: from dissemination to organ-specific colonization. Nat Rev Cancer 9 (4), 274-284 (2009); Pantel et al., Cancer micrometastases. Nat Rev Clin Oncol 6 (6), 339-351 (2009)).

It has been hypothesized that metastasis-initiating cells (MICs) are present within circulating-tumor-cells (CTCs) in the blood stream of carcinoma patients (Pantel et al., Detection, clinical relevance and specific biological properties of disseminating tumour cells. Nat Rev Cancer 8 (5), 329-340 (2008); Yu et al., Circulating tumor cells: approaches to isolation and characterization. J Cell Biol 192 (3), 373-382 (2011)). Indeed, the presence of CTCs in metastatic patients correlates with decreased overall-survival in several malignancies, including breast cancer (Cristofanilli et al., Circulating tumor cells, disease progression, and survival in metastatic breast cancer. N Engl J Med 351 (8), 781-791 (2004); Krebs et al., Evaluation and prognostic significance of circulating tumor cells in patients with non-small-cell lung cancer. J Clin Oncol 29 (12), 1556-1563 (2011); de Bono et al., Circulating tumor cells predict survival benefit from treatment in metastatic castration-resistant prostate cancer. Clin Cancer Res 14 (19), 6302-6309 (2008); Cohen et al., Prognostic significance of circulating tumor cells in patients with metastatic colorectal cancer. Ann Oncol 20 (7), 1223-1229 (2009); Hou et al., Clinical Significance and Molecular Characteristics of Circulating Tumor Cells and Circulating Tumor Microemboli in Patients With Small-Cell Lung Cancer. J Clin Oncol (2012)).

The detection and molecular characterization of CTCs in cancer patients could in principle allow unique insights into the development and progression of metastasis, the leading cause of cancer-related death. However, CTCs are extremely rare in samples obtained from patients (Pantel et al., Nat Rev Clin Oncol, loc. cit.; Yu et al., loc. cit.). As a consequence, their activity remains unexplored.

Thus, although the clinical data referred to above are consistent with the hypothesis that CTCs contain MICs, their existence, phenotype and activity has not been demonstrated. Therefore, there is a large unmet need in the diagnostic and therapeutic field for novel approaches in identifying and characterizing MICs in patient samples.

### OBJECTS OF THE INVENTION

It was an object of the invention to provide a method for the determining the presence of metastasis-initiating cells in circulating tumor cells, in particular in CTCs derived from breast cancer. Such a method, and the development of methods and composition for diagnostic and therapeutic applications, would offer major advantages for improving the treatment options for cancer patients.

### SUMMARY OF THE INVENTION

Surprisingly it has been found that a subpopulation of CTCs can be identified that are characterized by the simultaneous presence of c-MET, CD44 and CD47. Cell populations comprising such a subpopulation of OTCs result in the induction of metastasis in mouse models, which indicates that such CTC subpopulation comprises metastasis-initiating cells.

Thus, in one aspect, the present invention relates to a method for analyzing circulating tumor cells of a patient for the presence of metastasis-initiating cells comprising the step of detecting cells exhibiting the simultaneous presence of c-MET, CD44 and CD47.

In another aspect, the present invention relates to a kit for analyzing circulating tumor cells of a patient for the presence of metastasis-initiating cells comprising at least two reagents selected from:
(a) an analyte for detecting the presence of c-MET;
(b) an analyte for detecting the presence of CD44, and
(c) an analyte for detecting the presence of CD47.

In another aspect, the present invention relates to a kit for analyzing circulating tumor cells of a patient for the presence of metastasis-initiating cells comprising at least two reagents selected from:
(a) an analyte for detecting the presence of c-MET on the surface of circulating tumor cells;
(b) an analyte for detecting the presence of CD44 on the surface of circulating tumor cells; and
(c) an analyte for detecting the presence of CD47 on the surface of circulating tumor cells.

In yet another aspect, the present invention relates to a method for treating a patient having circulating tumor cells exhibiting the simultaneous presence of c-MET, CD44 and CD47 comprising the step of administering one or more pharmaceutical compositions targeting at least two proteins selected from c-MET, CD44 and CD47.

In a further aspect, the present invention relates to a bifunctional composition comprising targeting moieties for at least two proteins selected from c-MET, CD44 and CD47.

In a further aspect, the present invention relates to a bifunctional composition comprising targeting moieties for at least two proteins selected from c-MET, CD44 and CD47 for use in treating a patient having circulating tumor cells exhibiting the simultaneous presence of c-MET, CD44 and CD47.

In another aspect, the present invention relates to a kit for targeting circulating tumor cells of a patient exhibiting the simultaneous presence of c-MET, CD44 and CD47 comprising at least two pharmaceutical compositions selected from:
(a) a pharmaceutical composition for targeting c-MET;
(b) a pharmaceutical composition for targeting CD44, and
(c) a pharmaceutical composition for targeting CD47.

In another aspect, the present invention relates to a kit for targeting circulating tumor cells of a patient exhibiting the simultaneous presence of c-MET, CD44 and CD47 comprising at least two pharmaceutical compositions selected from (a) a pharmaceutical composition for targeting c-MET on the surface of circulating tumor cells;
(b) a pharmaceutical composition for targeting CD44on the surface of circulating tumor cells; and
(c) a pharmaceutical composition for targeting CD47on the surface of circulating tumor cells.

In a further aspect, the present invention relates to a method for stratification of patients having luminal breast cancer, comprising the step of detecting the presence or absence of CD47 on primary tumors, wherein the presence of CD47 defines patients with metastatic disease, and the absence of CD47 defines patients with non-metastatic disease.

In a further aspect, the present invention relates to a method for assessing breast cancer patient outcome, comprising the step of detecting c-MET expression on primary tumors, wherein the presence of c-MET cells defines patients with lower overall-survival probability, and the absence of c-MET defines patients with higher overall-survival probability.

In a further aspect, the present invention relates to a population of human tumor cells enriched for the presence of human tumor cells exhibiting the simultaneous presence of c-MET, CD44 and CD47.

### SUMMARY OF THE INVENTION

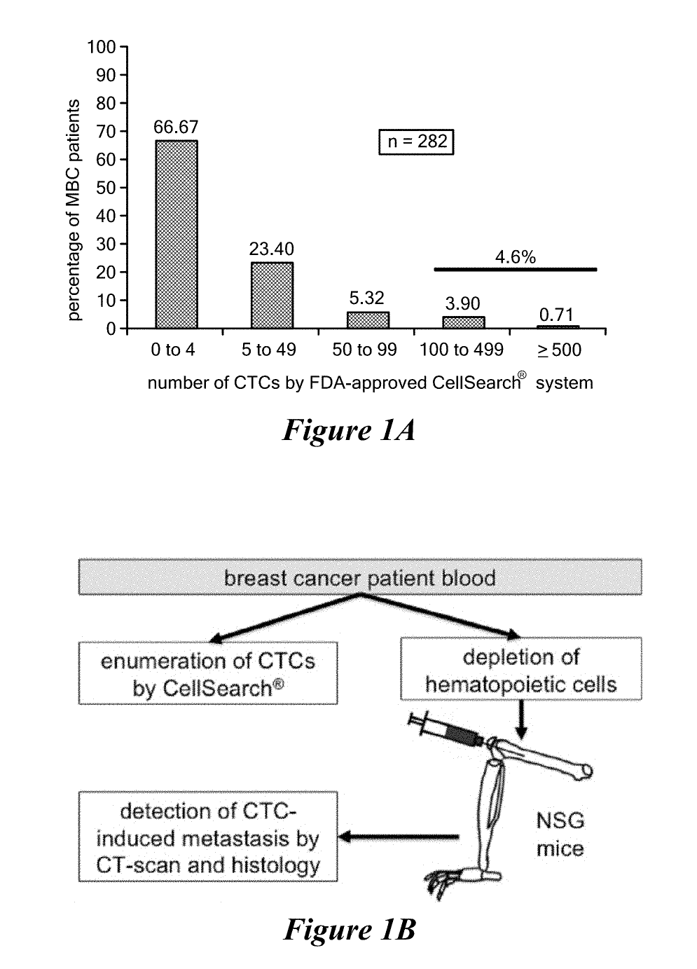

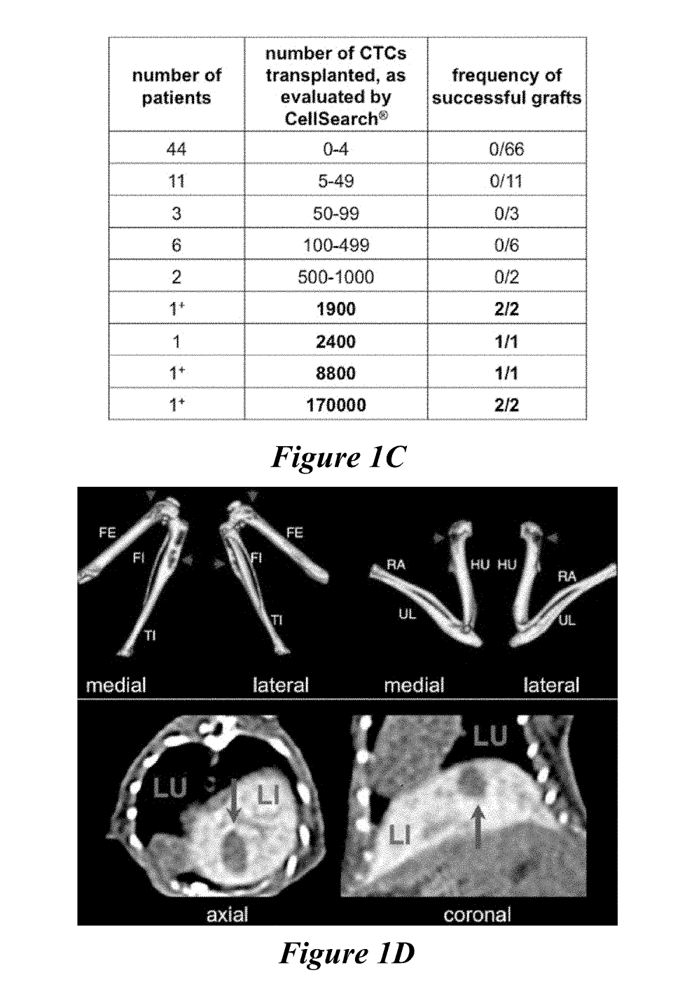

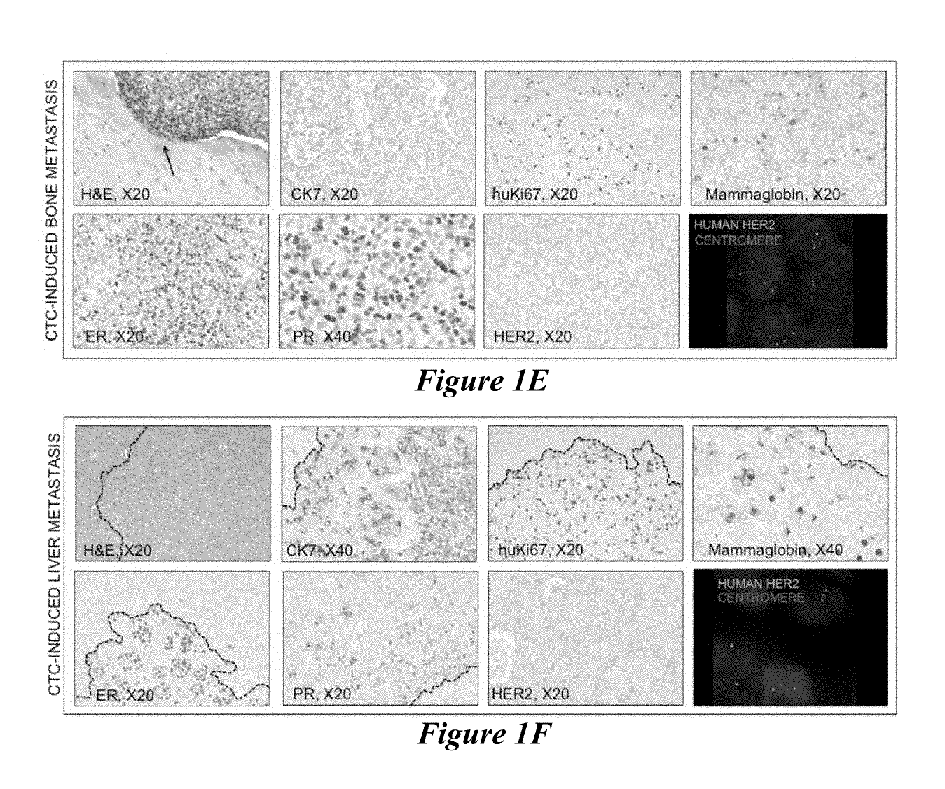

FIGS. 1A-1F show that rare patient CTCs contain MICs at low frequencies. FIG. 1A: distribution of the number of CTCs/7.5 mL blood within 282 metastatic breast cancer patients (MBC), as evaluated by CellSearch®. FIG. 1B: schematic representation of the MIC assay. Briefly, patient blood samples were enumerated by CellSearch® and in parallel were depleted of hematopoietic cells and transplanted into the femoral medullary cavity of NOD/SCID/IL2Ryc−/− (NSG) mice; FIG. 1C: results of the MIC assay: comparison of CTC numbers in patient peripheral blood samples as determined by CellSearch® versus the frequency of successful engraftment of tumors in mice. Stars indicate samples originating from patient#1, collected at different time points; FIG. 1D: volumetric-computed tomography (VCT) reconstructions of the long bones (left panel) and of the liver (right panel) of a representative mouse transplanted with CTCs isolated from patient#1 Left panel: VCT 3D volume rendering of long bones; left, medial and lateral views of the left hind leg (comprising the injected femur); right, medial and lateral views of the right forelimb; arrowheads: osteolytic lesions caused by bone metastases. Right panel: VCT sections of the liver after intravenous application of a liverspecific contrast agent; arrows point to liver metastasis; FIG. 1E: Patient#1 CTCinduced bone metastases in mice: H&E, CK7, human Ki67, mammaglobin, ER, PR and HER2 immunohistochemical stainings as well as human HER2 FISH analysis; FIG. 1F: Same analysis as in e, on CTC-induced liver metastases. Abbreviations: CTC: circulating tumor cells; CT: computed tomography; H&E: Hematoxylin and Eosin; CK7: Cytokeratin 7; FE: Femur; FI: Fibula; TI: Tibia; RA: Radius; HU: Humerus; UL: Ulna; LU: lung; LI: Liver; huKi67: human Ki67; ER: Estrogen Receptor; PR: Progesterone Receptor.

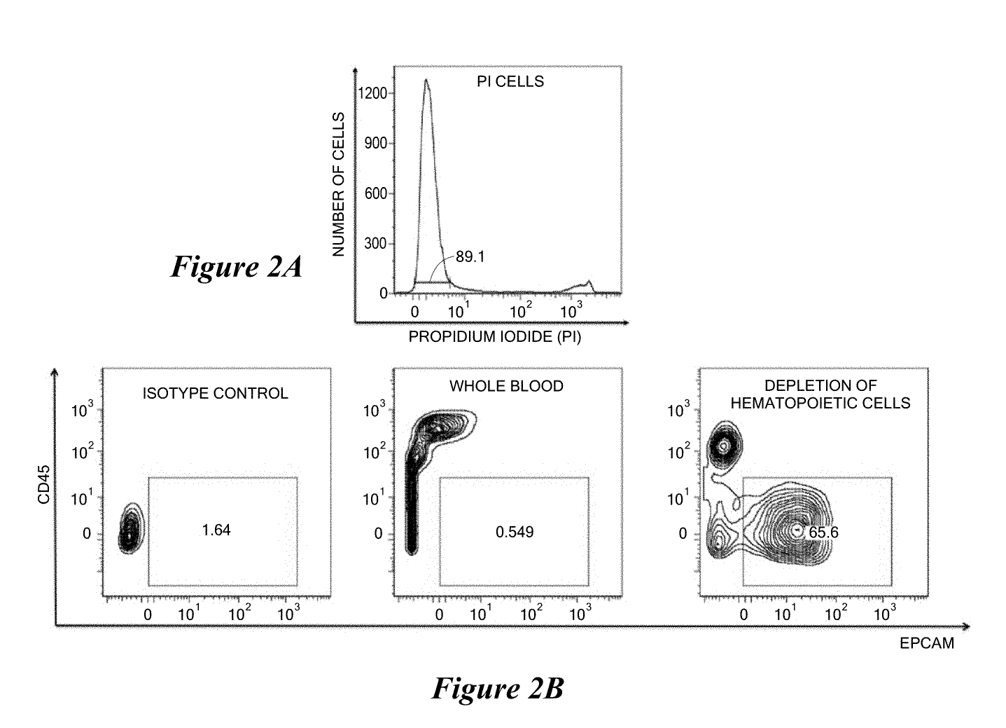

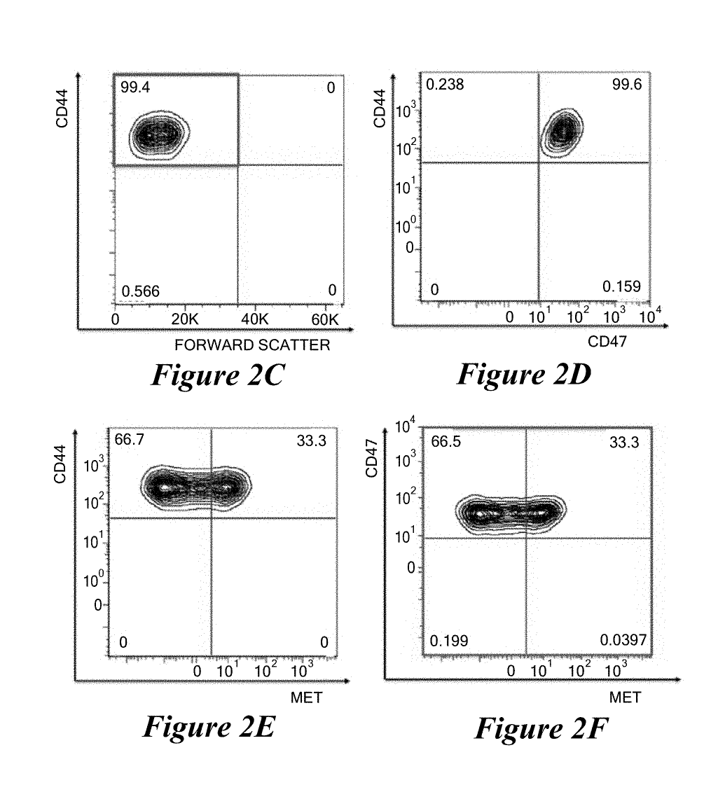

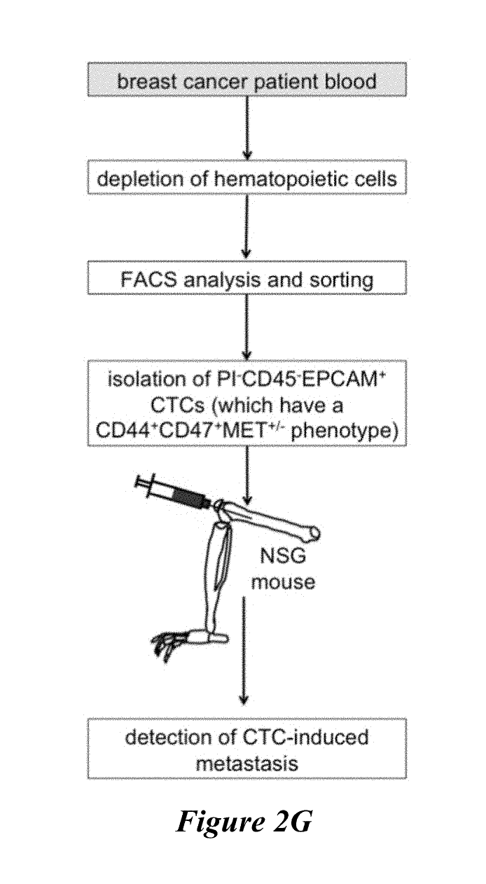

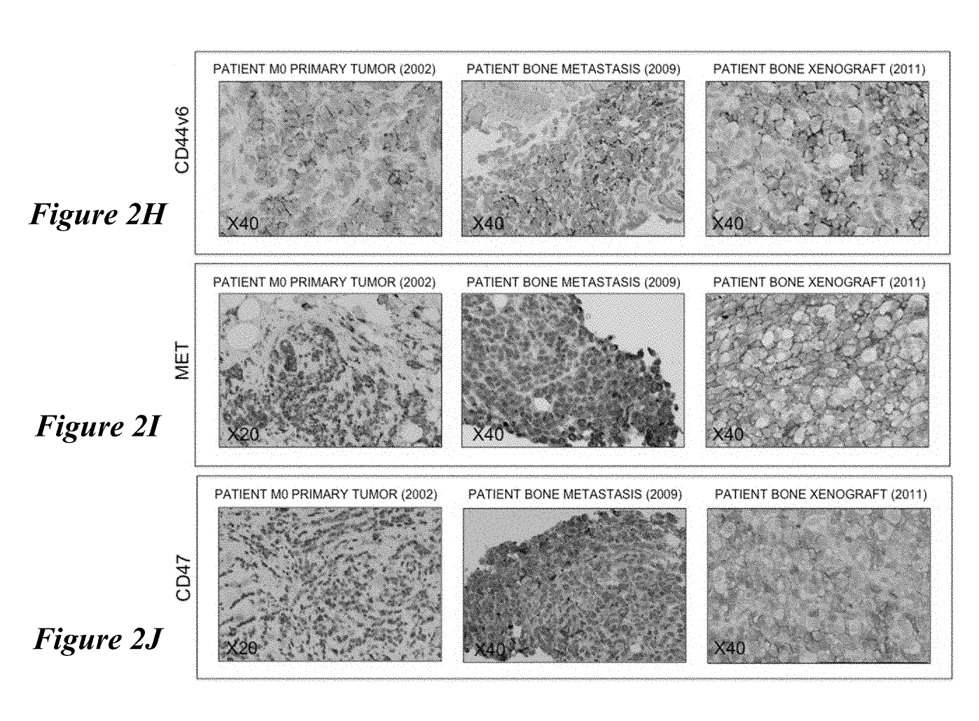

FIGS. 2A-2J show that luminal MICs express EPCAM, CD44, CD47 and possibly c-MET; FIGS. 2A-2F: FACS analysis of CTCs of patient#1; FIG. 2A: Gating strategy to select living cells (PI negative); FIG. 2B: EPCAM+−CTCs (defined as PI-CD45-EPCAM+), FIGS. 2C, 2D, and 2E: Expression of CD44, CD47 and c-MET on EPCAM+−CTCs, as indicated; FIG. 2F: Expression of CD47 and c-MET on CD44+EPCAM+−CTCs; FIG. 2G: experimental outline used for h-j; FIG. 2H: Expression of CD44v6; FIG. 2I: c-MET; FIG. 2J: CD47, in the non-metastatic primary tumor (collected in 2002), bone metastasis (collected in 2009) and EPCAM+CD44+CD47+CTC-induced metastasis in mice of patient#1.

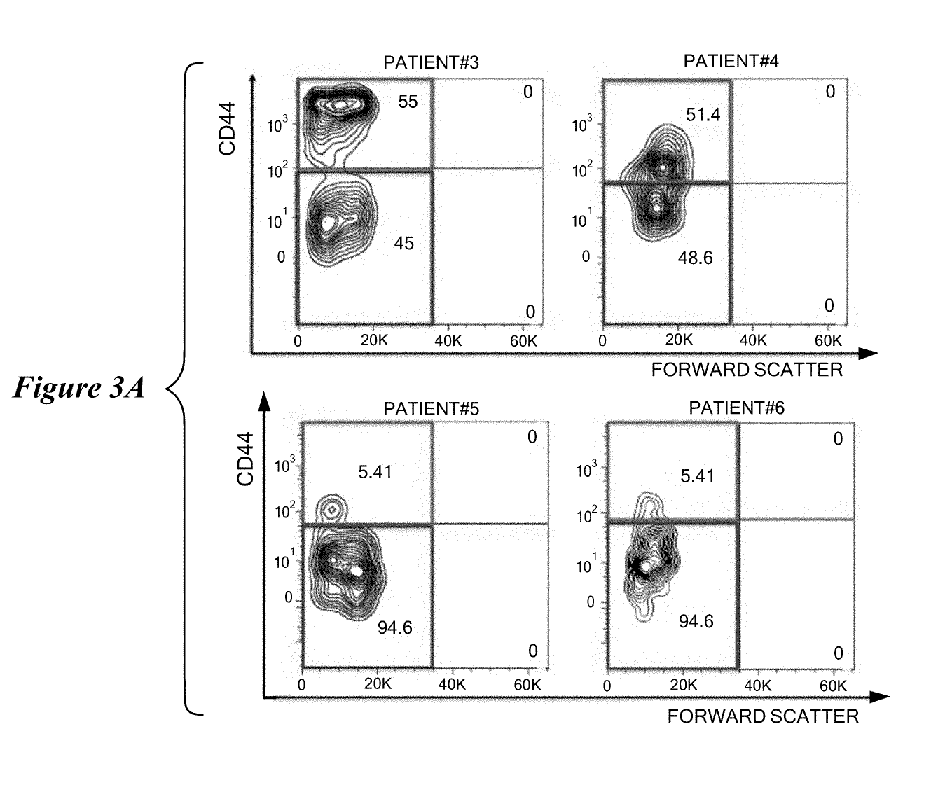

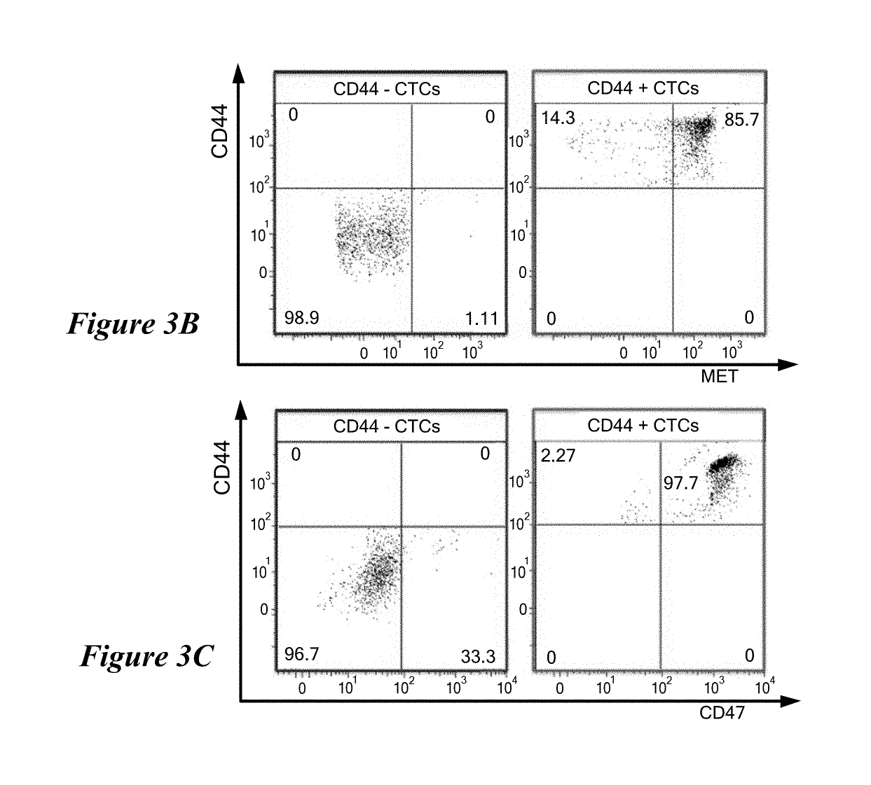

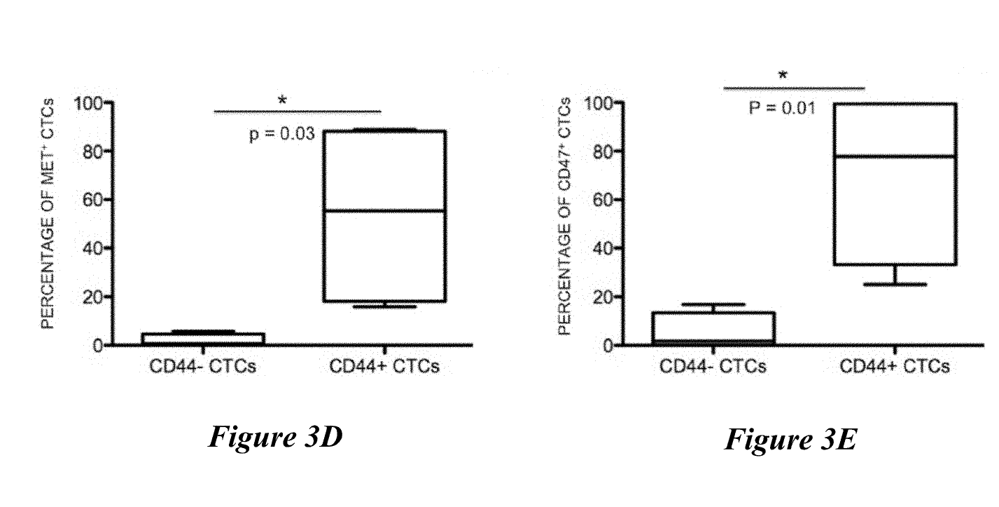

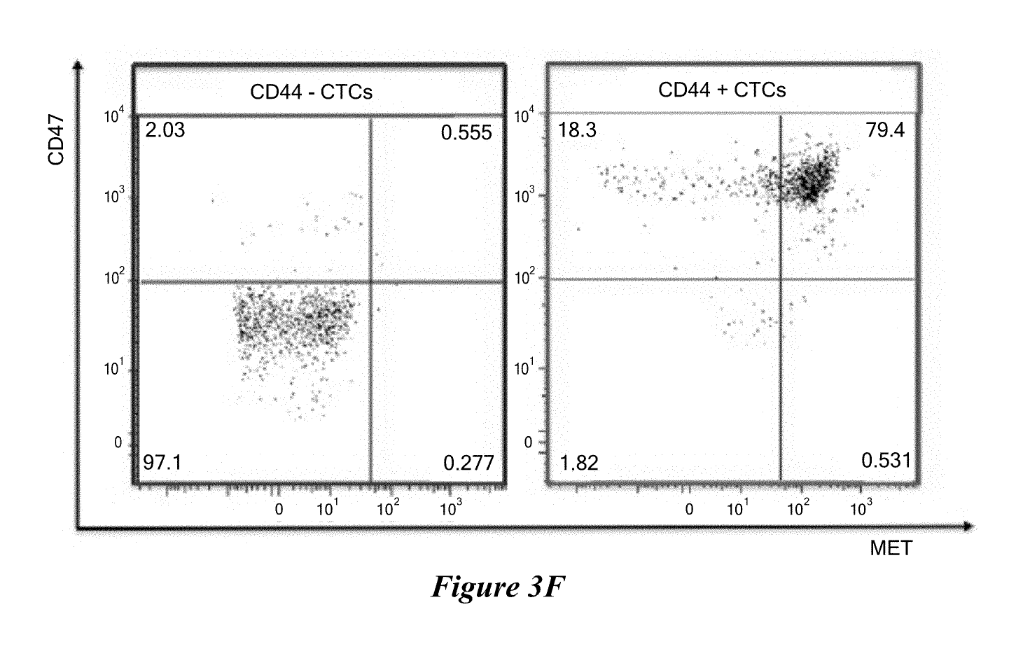

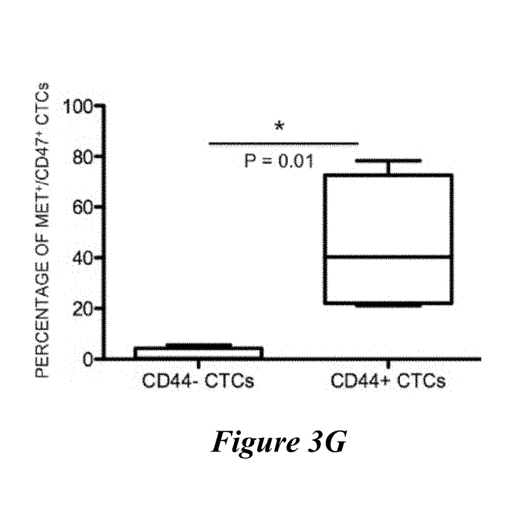

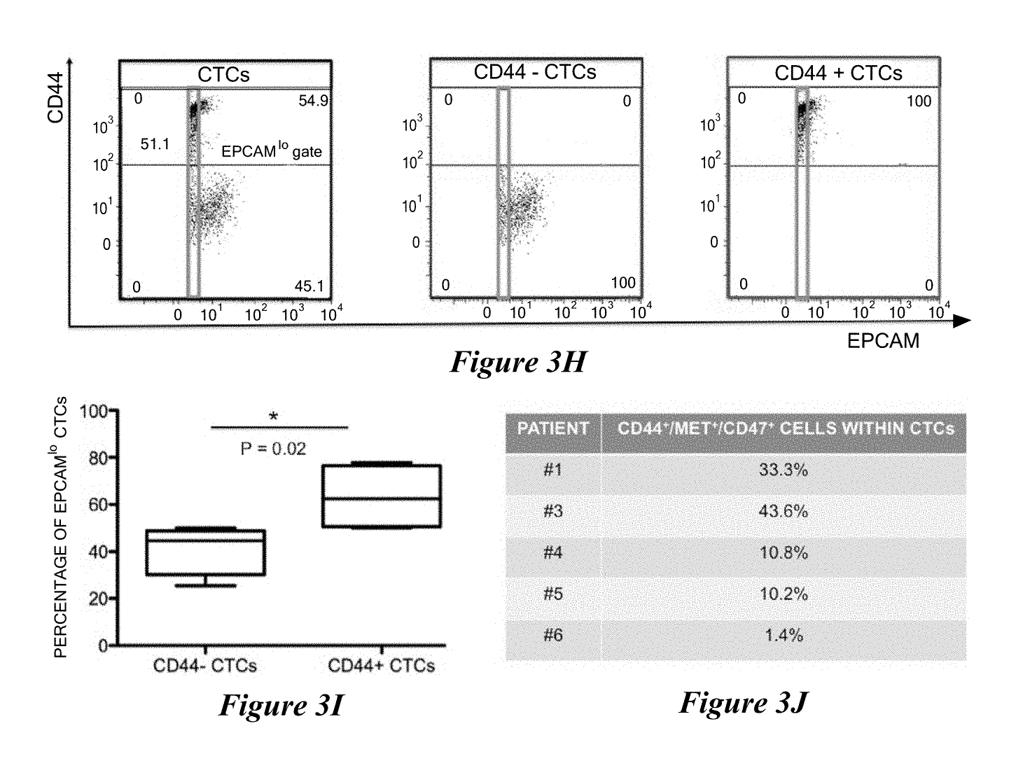

FIGS. 3A-3J show the expression of CD44, CD47 and c-MET in luminal patient CTCs. FIG. 3A: Expression of CD44 in EPCAM+−CTCs (defined as PI-CD45-EPCAM+, appearing simply as “CTCs” in this figure) of patients #3-6. Red: CD44+EPCAM+−CTCs; Blue: CD44− EPCAM+−CTCs. Patient#3: c-MET expression FIG. 3B, CD47 expression FIG. 3C, CD47 and c-MET co-expression, EPCAMₗₒ gate and EPCAMₗₒ expression FIG. 3H, within CD44+ and CD44− EPCAM+−CTCs; FIG. 3F: Mean percentage of c-MET-expressing cells FIG. 3D, CD47-expressing cells FIG. 3E, CD47+/c-MET+ cells FIG. 3G, and EPCAMₗₒ cells i, within CD44+ and CD44− EPCAM+−CTCs of patients #3-6; FIG. 3J: Frequencies of CD44+/c-MET+/CD47+ cells within EPCAM+−CTCs. P-values were calculated using an unpaired Student T-test. Abbreviations: CTC: Circulating Tumor Cell; EPCAM: Epithelial Cell Adhesion Molecule; PI: Propidium Iodide.

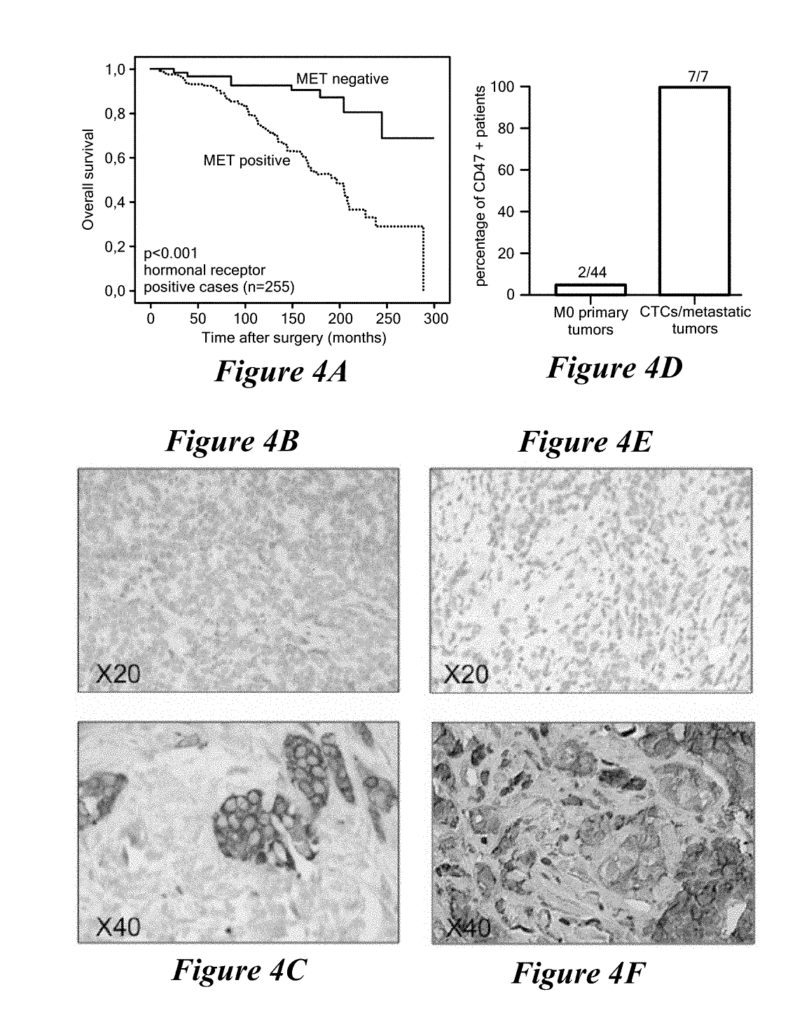

FIGS. 4A-4F show that c-MET expression serves as a prognostic factor for overall-survival and CD47 expression is specific for metastatic tumor cells of hormonal receptor positive patients. FIG. 4A: Kaplan Meier survival analysis of 255 hormonal receptor positive (HR+) breast cancer patients in relation to c-MET expression. Representative tumors that were negative, FIG. 4B, or positive, FIG. 4C, for c-MET expression; FIG. 4D: Percentage of HR+ patients expressing CD47 in non-metastatic (MO) primary tumors (n=44) compared to expression in CTCs and/or metastatic lesions (n=7). Representative CD47 negative non-metastatic primary tumor FIG. 4E, or a CD47 positive metastatic tumor FIG. 4F.

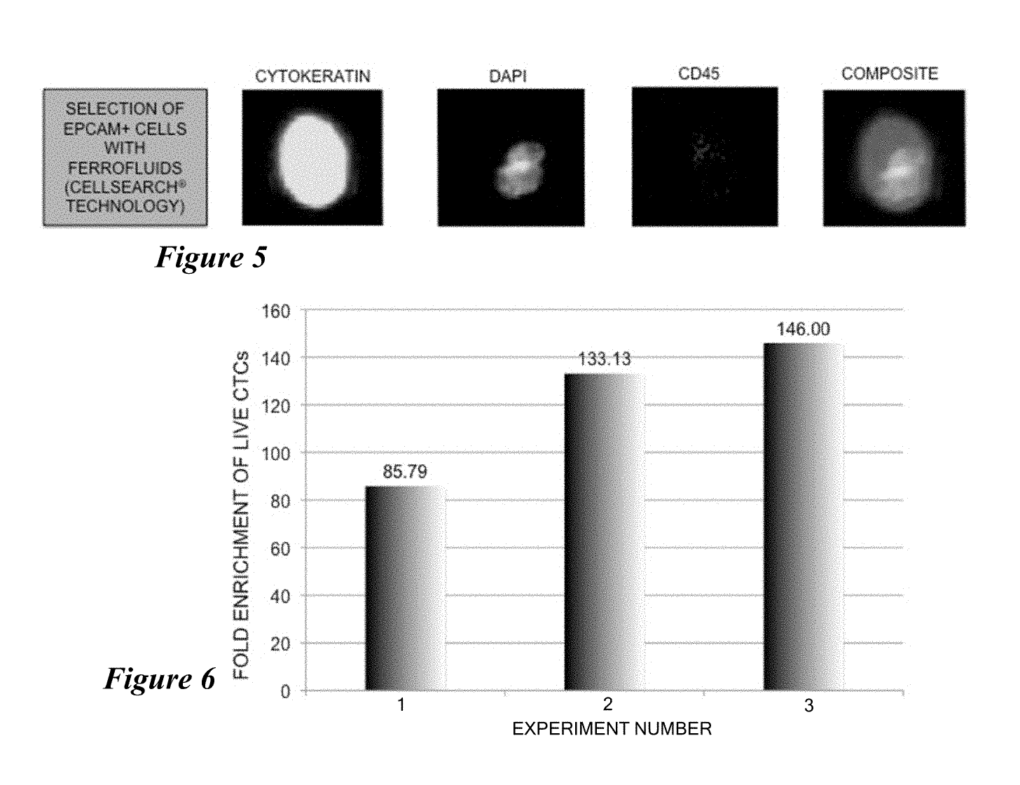

FIG. 5 shows the results of a CellSearch®-mediated detection of patient circulating tumor cells. Example of a CTC detected by CellSearch®. Captured by EPCAM-antibody-bearing ferrofluids, it is positive for cytokeratins (epithelial marker), showing an intact nucleus (detected by DAPI staining), but is negative for the common leukocyte antigen CD45. Abbreviations: DAPI: 4′,6-diamidino-2-phenylindole; EPCAM: Epithelial Cell Adhesion Molecule CTCs: circulating tumor cells.

FIG. 6 shows the degree of enrichment of patient CTCs after RosetteSep® depletion. Patient EPCAM+−CTCs were detected by FACS analysis, before and after enrichment with RosetteSep®. The enrichment is shown for 3 independent experiments. Abbreviations: CTCs: Circulating Tumor Cells.

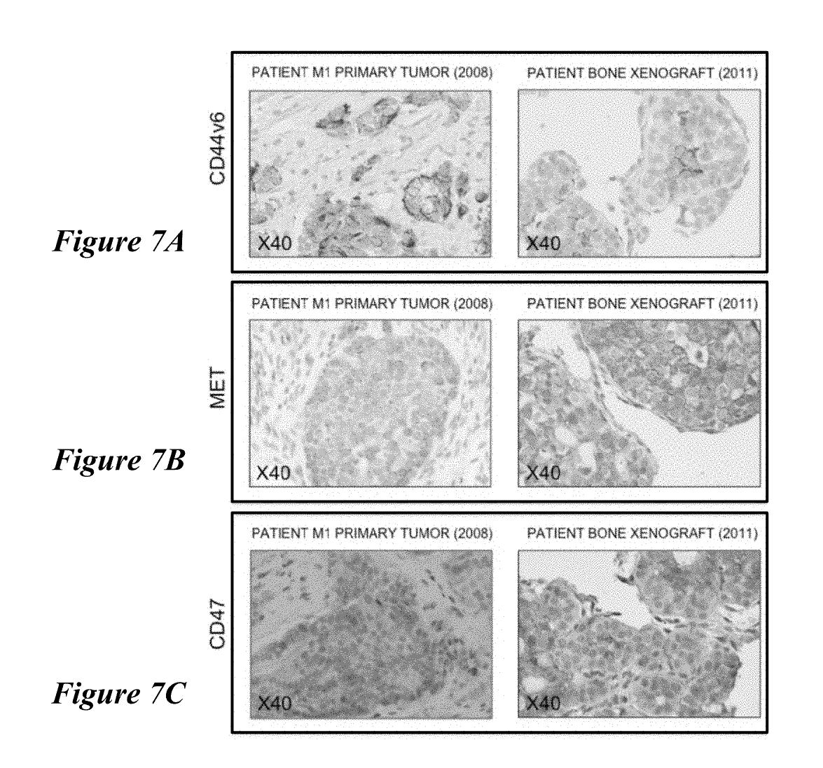

FIGS. 7A-7C shows the analysis of CD44v6 (FIG. 7A), c-MET (FIG. 7B) and CD47 (FIG. 7C) expression in bone metastasis induced in mice by OTCs isolated from patient#2.

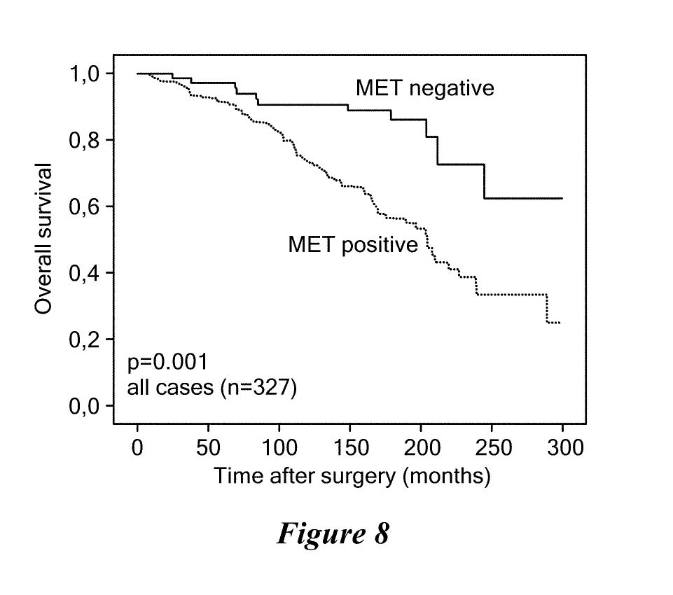

FIG. 8 shows the Kaplan Meier survival analysis of 327 breast cancer patients (whole tissue-microarray cohort), according to c-MET expression.

### DETAILED DESCRIPTION OF THE INVENTION

The present invention may be understood more readily by reference to the following detailed description of the invention and the examples included therein.

Thus, in one aspect, the present invention relates to a method for analyzing circulating tumor cells of a patient for the presence of metastasis-initiating cells comprising the step of detecting cells exhibiting the simultaneous presence of c-MET, CD44 and CD47.

In the context of the present invention, the term “circulating tumor cell” refers to viable tumor cells disseminated from a primary tumor of a patient that are found in the blood of that patient.

Methods for analyzing cells found in the blood of a patient and for identifying and determining circulating tumor cells are well known for one of ordinary skill in the art, and include, for example, staining with propidium iodide (PI) in order to determine the viability of cells (PI⁻: viable), and exclusion of cells normally found in blood by staining for cells surface antigens characteristic for hematopoietic cells (e.g. CD45). Depending on the type of primary tumor, an additional analysis may be performed by staining for cells surface antigens characteristic for the primary tumor (e.g. EPCAM for tumors of epithelial origin, including breast cancer). The number of OTCs may, for example, be determined using the FDA-approved CellSearch® system (Veridex, Raritan, N.J., USA).

In the context of the present invention, the term “comprises” or “comprising” means “including, but not limited to”. The term is intended to be open-ended, that specify the presence of any stated features, elements, integers, steps, or components, but do not preclude the presence or addition of one or more other features, elements, integers, steps, components, or groups thereof. The term “comprising” thus includes the more restrictive terms “consisting of” and “consisting essentially of”.

In the context of the present invention, the presence of c-MET, CD44 and/or CD47 may be detected in any way known to one of ordinary skill in the art, including the direct detection of the protein, either inside a cell, presented at the surface of a cell, or secreted from a cell, and further including the detection of a nucleic acid encoding at least a part of the protein, including genomic DNA and mRNA transcripts.

In particular embodiments, variants of c-MET, CD44 and/or CD37 are detected, such as mutated forms, isoforms or splice variants, either alternatively or additionally. In particular embodiments, the CD44v6 isoform is detected.

In certain embodiments of the present invention, said patient has breast cancer.

In gene expression studies it has been found that breast cancer can be grouped in five breast cancer subtypes: luminal A/B, HER 2 overexpressing, basal-like, and unclassified.

Luminal tumors are characterized by expression of hormonal receptors, particularly the estrogen receptor, which is usually accompanied by expression of the progesterone receptor. In HER 2 overexpressing tumors the protein kinase HER-2/neu is upregulated. Basal-like and unclassified tumors are negative for these three receptors.

In certain such embodiments, said breast cancer is hormonal receptor positive breast cancer.

In certain embodiments of the present invention, said presence of c-MET, CD44 and CD47 is detected in sequential order.

In certain such embodiments, c-MET, CD44 or CD47 is detected as protein.

In particular embodiments, c-MET, CD44 or CD47 is detected on the surface of a circulating tumor cell.

In certain such embodiments, a nucleic acid encoding c-MET, CD44 or CD47 is detected.

In particular embodiments, an mRNA encoding c-MET, CD44 or CD47 is detected.

In certain such embodiments, fluorescence-assisted cell sorting is used.

In certain embodiments of the present invention, said presence of at least two of c-MET, CD44 and CD47 is detected in parallel.

In certain such embodiments, c-MET, CD44 or CD47 is detected as protein.

In certain such embodiments, a nucleic acid encoding c-MET, CD44 or CD47 is detected.

In certain such embodiments, a multiplexing approach is used.

In the context of the present application, a “multiplexing approach” is defined as an approach, wherein at least two individual parameters are determined in one step, e.g. by simultaneously determining the presence of two or more surface protein using differentially labeled analytes by using, for example, confocal laser scanning microscopy.

In another aspect, the present invention relates to a kit for analyzing circulating tumor cells of a patient for the presence of metastasis-initiating cells comprising at least two reagents selected from:
(a) an analyte for detecting the presence of c-MET;
(b) an analyte for detecting the presence of CD44, and
(c) an analyte for detecting the presence of CD47.

In the context of the present invention, an analyte for detecting one of the proteins listed may be a reagent detecting the presence of c-MET, CD44 or CD47 as protein, including embodiments where the analyte is selected from: a natural ligand for c-MET, CD44 or CD47, a mimic of a natural ligand for c-MET, CD44 or CD47, or an antibody specific for c-MET, CD44 or CD47, or functional fragment of such an antibody.

In the context of the present invention, an analyte for detecting one of the proteins listed may alternatively be a reagent detecting the presence of a nucleic acid encoding c-MET, CD44 or CD47, including embodiments where the analyte is selected from: a nucleic acid hybridizing with the nucleic acid encoding c-MET, CD44 or CD47 under the conditions present inside the CTC, including DNA, RNA, and siRNA molecules.

In certain embodiments of the present invention, said kit comprises all three reagents (a) to (c).

In another aspect, the present invention relates to a kit for analyzing circulating tumor cells of a patient for the presence of metastasis-initiating cells comprising at least two reagents selected from:
(a) an analyte for detecting the presence of c-MET on the surface of circulating tumor cells;
(b) an analyte for detecting the presence of CD44 on the surface of circulating tumor cells; and
(c) an analyte for detecting the presence of CD47 on the surface of circulating tumor cells.

In the context of the present invention, an analyte for detecting one of the proteins listed may be a reagent detecting the presence of c-MET, CD44 or CD47 as protein, including embodiments where the analyte is selected from: a natural ligand for c-MET, CD44 or CD47, a mimic of a natural ligand for c-MET, CD44 or CD47, or an antibody specific for c-MET, CD44 or CD47, or functional fragment of such an antibody.

In certain embodiments of the present invention, said kit comprises all three reagents (a) to (c).

In certain embodiments of the present invention, one or more of said analytes are antibody-based analytes.

As used herein, the term “antibody” refers to an immunoglobulin (Ig) molecule that is defined as a protein belonging to the class IgG, IgM, IgE, IgA, or IgD (or any subclass thereof), which includes all conventionally known antibodies and functional fragments thereof. An “antibody-based analyte” is either a full antibody molecule or a functional fragment thereof. A “functional fragment” of an antibody/immunoglobulin molecule hereby is defined as a fragment of an antibody/immunoglobulin molecule (e.g., a variable region of an IgG) that retains the antigen-binding region. An “antigen-binding region” of an antibody typically is found in one or more hypervariable region(s) (or complementarity-determining region, “CDR”) of an antibody molecule, i.e. the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of antibody molecules for use in the present invention is IgG.

“Functional fragments” of the invention include the domain of a F(ab′)2 fragment, a Fab fragment, scFv or constructs comprising single immunoglobulin variable domains or single domain antibody polypeptides, e.g. single heavy chain variable domains or single light chain variable domains. The F(ab′)2 or Fab may be engineered to minimize or completely remove the intermolecular disulphide interactions that occur between the CH1 and CL domains.

An antibody may be derived from immunizing an animal, or from a recombinant antibody library, including an antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67; and U.S. Pat. No. 6,300,064 issued to Knappik et al.

In certain such embodiments, all three of said analytes are antibody-based analytes.

In yet another aspect, the present invention relates to a method for treating a patient having circulating tumor cells exhibiting the simultaneous presence of c-MET, CD44 and CD47 comprising the step of administering one or more pharmaceutical compositions targeting at least two proteins selected from c-MET, CD44 and CD47.

In the context of the present invention, a pharmaceutical composition targeting at least two proteins selected from c-MET, CD44 and CD47 may target c-MET, CD44 and/or CD47 in any way known to one of ordinary skill in the art, including the direct targeting of the protein, either inside a cell, or presented at the surface of a cell, or targeting of a nucleic acid encoding at least a part of the protein, including genomic DNA and mRNA transcripts.

In another aspect, the present invention relates to a kit for targeting circulating tumor cells of a patient exhibiting the simultaneous presence of c-MET, CD44 and CD47 comprising at least two pharmaceutical compositions selected from:
(a) a pharmaceutical composition for targeting c-MET;
(b) a pharmaceutical composition for targeting CD44, and
(c) a pharmaceutical composition for targeting CD47.

In another aspect, the present invention relates to a kit for targeting circulating tumor cells of a patient exhibiting the simultaneous presence of c-MET, CD44 and CD47 comprising at least two pharmaceutical compositions selected from (a) a pharmaceutical composition for targeting c-MET on the surface of circulating tumor cells;
(b) a pharmaceutical composition for targeting CD44on the surface of circulating tumor cells; and
(c) a pharmaceutical composition for targeting CD47on the surface of circulating tumor cells.

In particular embodiments, variants of c-MET, CD44 and/or CD37 are targeted, such as mutated forms, isoforms or splice variants, either alternatively or additionally. In particular embodiments, the CD44v6 isoform is targeted.

In certain embodiments of the present invention, a pharmaceutical composition is administered, which comprises binding sites for at least two proteins selected from c-MET, CD44 and CD47.

In certain embodiments of the present invention, said pharmaceutical composition is a bispecific antibody or functional bispecific fragment thereof.

In the context of the present invention, the term “bispecific antibody molecule” refers to an antibody molecule, including a functional fragment of an antibody molecule, that comprises specific binding sites for two different targets, biomolecules, or two different epitopes, either present on one target biomolecule, or present on two different molecules, such as on the target biomolecule and a second biomolecule.

As used herein, a binding molecule is “specific to/for”, “specifically recognizes”, or “specifically binds to” a target, such as a target biomolecule (or an epitope of such biomolecule), when such binding molecule is able to discriminate between such target biomolecule and one or more reference molecule(s), since binding specificity is not an absolute, but a relative property. In its most general form (and when no defined reference is mentioned), “specific binding” is referring to the ability of the binding molecule to discriminate between the target biomolecule of interest and an unrelated biomolecule, as determined, for example, in accordance with a specificity assay methods known in the art. Such methods comprise, but are not limited to Western blots, ELISA, RIA, ECL, IRMA tests and peptide scans. For example, a standard ELISA assay can be carried out. The scoring may be carried out by standard colour development (e.g. secondary antibody with horseradish peroxide and tetramethyl benzidine with hydrogen peroxide). The reaction in certain wells is scored by the optical density, for example, at 450 nm. Typical background (=negative reaction) may be about 0.1 OD; typical positive reaction may be about 1 OD. This means the ratio between a positive and a negative score can be 10-fold or higher. Typically, determination of binding specificity is performed by using not a single reference biomolecule, but a set of about three to five unrelated biomolecules, such as milk powder, BSA, transferrin or the like.

In the context of the present invention, the term “about” or “approximately” means between 90% and 110% of a given value or range.

However, “specific binding” also may refer to the ability of a binding molecule to discriminate between the target biomolecule and one or more closely related biomolecule(s), which are used as reference points. Additionally, “specific binding” may relate to the ability of a binding molecule to discriminate between different parts of its target antigen, e.g. different domains, regions or epitopes of the target biomolecule, or between one or more key amino acid residues or stretches of amino acid residues of the target biomolecule.

In certain embodiments of the present invention, said patient has breast cancer.

In certain such embodiments, said breast cancer is hormonal receptor positive breast cancer.

In the context of the present invention, the term “pharmaceutical composition” refers to compositions for use in humans, which comprise the therapeutically active ingredient, and optionally a pharmaceutically acceptable carrier and/or excipient. The compositions may be formulated e.g. for once-a-day administration, twice-a-day administration, or three times a day administration.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an active compound (e.g., a bispecific antibody fragment) is administered. Such pharmaceutical carriers may be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by A. R. Gennaro, 20th Edition.

The active ingredient (e.g., the bispecific antibody molecule or fragment thereof) or the composition of the present invention may be used for the manufacture of a medicament for the treatment of at least one disease or disorder, wherein the medicament is adapted to or appropriately prepared for a specific administration as disclosed herein (e.g., to once-a-day, twice-a-day, or three times a day administration). For this purpose the package leaflet and/or the patient information contains corresponding information.

In a further aspect, the present invention relates to a bifunctional composition comprising targeting moieties for at least two proteins selected from c-MET, CD44 and CD47.

In certain embodiments of the present invention, said bifunctional composition is a bispecific antibody or functional bispecific fragment thereof comprising binding sites for at least two antigens selected from c-MET, CD44 and CD47.

In the context of the present invention, the term “functional bispecific fragment thereof” refers to an antibody-based molecule that does not comprise all variable and constant domains of a full antibody in their entire length, provided that at least those parts of a full antibody are maintained that are required for providing the binding sites for the two antigens the bispecific antibody is specific for. Examples of such bispecific
fragments are a. Diabodies (Perisic et al., Structure. 1994 Dec. 15; 2(12):1217-26; Kontermann, Acta Pharmacol Sin. 2005 January; 26(1):1-9; Kontermann, Curr Opin Mol Ther. 2010 April; 12(2):176-83.)
b. TandAbs etc. (Cochlovius et al., Cancer Res. 2000 Aug. 15; 60(16):4336-41.)
c. Single domains specific to different targets genetically fused by peptide linkers (e.g. Domantis: WO/2008/096158, Ablynx: WO/2007/112940)
d. Others (for reviews, see: Enever et al., Curr Opin Biotechnol. 2009 August; 20(4):405-11. Epub 2009 Aug. 24.; Carter, Nat. Rev. Immunol. 6, 343 (2006); P. Kufer et al., Trends Biotechnol. 22, 238 (2004)).

In a further aspect, the present invention relates to a bifunctional composition comprising targeting moieties for at least two proteins selected from c-MET, CD44 and CD47 for use in treating a patient having circulating tumor cells exhibiting the simultaneous presence of c-MET, CD44 and CD47.

In certain embodiments of the present invention, said bifunctional composition is a bispecific antibody or functional bispecific fragment thereof comprising binding sites for at least two antigens selected from c-MET, CD44 and CD47.

In certain embodiments of the present invention, said patient has breast cancer.

In certain such embodiments, said breast cancer is hormonal receptor positive breast cancer.

In a further aspect, the present invention relates to a method for stratification of patients having luminal breast cancer, comprising the step of detecting the presence or absence of CD47 on primary tumors, wherein the presence of CD47 defines patients with metastatic disease, and the absence of CD47 defines patients with non-metastatic disease.

In the context of the present invention, the term “stratification” refers to the classification or grouping of patients according to one or more predetermined criteria. In certain embodiments, stratification is performed in a diagnostic setting in order to group a patient according to the prognosis of disease progression, either with or without treatment. In particular embodiments, stratification is used in order to distribute patients enrolled for a clinical study according to their individual characteristics. In particular embodiments, stratification is used in order to identify the best suitable treatment option for a patient.

In a further aspect, the present invention relates to a method for assessing breast cancer patient outcome, comprising the step of detecting c-MET expression on primary tumors, wherein the presence of c-MET cells defines patients with lower overall-survival probability, and the absence of c-MET defines patients with higher overall-survival probability.

In certain embodiments, said breast cancer is hormonal receptor positive breast cancer.

In a further aspect, the present invention relates to a population of human tumor cells enriched for the presence of human tumor cells exhibiting the simultaneous presence of c-MET, CD44 and CD47.

In certain embodiments, said population of human tumor cells comprises more than 50%, 75%, or 90% human tumor cells which exhibit the simultaneous presence of c-MET, CD44 and CD47.

In certain embodiments, said population of human tumor cells comprises more than 50%, 75%, or 90% human tumor cells which exhibit the simultaneous presence of c-MET, CD44 and CD47.

A population of human tumor cells enriched for the presence of human tumor cells exhibiting the simultaneous presence of c-MET, CD44 and CD47 may be obtained, for example, by fluorescence-assisted cell sorting, or by using immobilized capturing agents.

The invention is now described with reference to the following examples. These examples are provided for the purpose of illustration only and the invention should not be construed as being limited to these examples, but rather should be construed to encompass any and all variations which become evident as a result of the teaching provided herein. The following materials and methods are provided with respect to the subsequent examples but do not limit a multiplicity of materials and methodologies encompassed by the present invention.

### EXAMPLES

### Example: Metastasis-Initiating Cells are Present within Breast Cancer EPCAM+CD44+CD47+ Circulating Tumor Cells

### SUMMARY

Here we show the existence of MICs among CTCs directly isolated from the blood of patients with hormonal receptor-positive (HR+) luminal breast cancer, the most common subtype of mammary malignancies (Herschkowitz et al., Identification of conserved gene expression features between murine mammary carcinoma models and human breast tumors. Genome Biol 8 (5), R76 (2007); Perou et al., Molecular portraits of human breast tumours. Nature 406 (6797), 747-752 (2000)).

As low as 1900 CTCs were able to induce metastases in mice and transplantation of CTCs with an EPCAM+CD44+CD47+ phenotype induced metastatic growth in bones and liver, demonstrating the presence of MICs. FACS analysis of primary patient EPCAM+−CTCs revealed heterogeneous inter-patient expression of the metastasis-promoting signaling receptors CD44, CD47 and c-MET. While the percentage of EPCAM+CD44+CD47+c-MET+CTCs varied between 1.4 and 44%, metastases both from the original patient and those derived experimentally from CTCs showed high levels of all three receptors.

Tissue-microarray analysis revealed that expression of the c-MET tyrosinekinase receptor in HR+ cancers is an independent predictor of decreased overall-survival (hazard ratio 4.7, p<0.001). Finally, while non-metastatic HR+ tumors typically did not express CD47, all metastatic patients expressed CD47 in CTCs and/or metastatic tissues, suggesting that CD47 is specifically up-regulated at the onset of the metastatic process. The data provide a first demonstration that EPCAM+−CTCs express CD44, CD47 and c-MET and contain MICs, providing a molecular basis for the design of diagnostic tools to detect MICs and for developing rational-based approaches to target metastasis in breast cancer.

### DESCRIPTION

In a cohort of 282 progressive metastatic breast cancer (MBC) patients in our comprehensive cancer center, only 4.6% displayed≥100 CTCs/7.5 mL as measured by the Food and Drug Administration (FDA)-approved CellSearch® system (Riethdorf et al., Detection of circulating tumor cells in peripheral blood of patients with metastatic breast cancer: a validation study of the CellSearch system. Clin Cancer Res 13 (3), 920-928 (2007)) (FIG. 1a, FIG. 5). In order to test whether metastasis-initiating cells (MICs) are present within CTCs, patient blood samples depleted of hematopoietic cells were transplanted into the femoral medullary cavity of immuno-compromised NOD/SCID/IL2Ryc−/− (NSG) mice (Ito et al., NOD/SCID/gamma(c)(null) mouse: an excellent recipient mouse model for engraftment of human cells. Blood 100 (9), 3175-3182 (2002) (FIG. 1b). 15 months after transplantation, the injection of up to 1000 CellSearch®-evaluated CTCs isolated from 66 different MBC patients did not lead to metastatic growth. In contrast, six recipient mice receiving at least 1900 CTCs isolated from two patients (#1 and #2) developed multiple bone and liver metastases after 6 to 12 months (FIG. 1c, Table 1). This became evident by CT-scanning and was confirmed by histology (FIG. 1d-f). Strikingly, bone tumors not only arose in injected-femurs, but also in other bones, generating typical osteolytic lesions (FIG. 1d). In addition, CTC-induced bone and liver metastases in mice displayed the same hormonal receptor status (ER+, PR+ and HER2−) as the patient primary tumors (FIG. 1e-f, Table 1). The human origin of the metastases was confirmed by human-specific FISH and by human-specific Ki67 expression (FIG. 1e-f). The breast tissue of origin was shown by expression of mammaglobin (FIG. 1e-f). To further analyze the phenotype of MIC-containing CTCs, hematopoietic celldepleted blood of patient#1 was analyzed by flow-cytometry (FACS): “EPCAM+− CTCs” were defined as cells negative both for Propidium-Iodide (PI, viable cells) and CD45 (exclusion of most hematopoietic cells), but positive for the epithelial marker EPCAM (FIG. 2a-b). After hematopoietic cell-depletion, EPCAM+−CTCs could be detected at >100-fold higher frequencies compared to un-fractionated patient blood, facilitating the generation of robust FACS data for patients with 100 CTCs (FIG. 2b, FIG. 6). First, expression of the cancer stem cell (CSC) marker and bone homing receptor CD44 was analyzed in EPCAM+−CTCs (Al-Hajj et al., Prospective identification of tumorigenic breast cancer cells. Proc Natl Acad Sci USA 100 (7), 3983-3988 (2003)). Strikingly, all EPCAM+−CTCs of patient#1 expressed CD44 (FIG. 2c). Second, CD47, which inhibits phagocytosis and has been implicated in the evasion of cancer cells from the innate immune system, was examined (Majeti, R. et al., CD47 is an adverse prognostic factor and therapeutic antibody target on human acute myeloid leukemia stem cells. Cell 138 (2), 286-299 (2009); Chao et al., Therapeutic antibody targeting of CD47 eliminates human acute lymphoblastic leukemia. Cancer Res 71 (4), 1374-1384 (2011); Chan et al., Identification, molecular characterization, clinical prognosis, and therapeutic targeting of human bladder tumorinitiating cells. Proc Natl Acad Sci USA 106 (33), 14016-14021 (2009): Chao et al., Anti-CD47 antibody synergizes with rituximab to promote phagocytosis and eradicate non-Hodgkin lymphoma. Cell 142 (5), 699-713 (2010).

All CD44+EPCAM+−CTCs also expressed CD47 (FIG. 2d). Third, the tyrosine kinase receptor c-MET, which has been suggested to activate a metastatic program (Trusolino et al., MET signalling: principles and functions in development, organ regeneration and cancer. Nat Rev Mol Cell Biol 11 (12), 834-848 (2010); Gentile et al., The Met tyrosine kinase receptor in development and cancer. Cancer Metastasis Rev 27 (1), 85-94 (2008), was expressed by 33% of EPCAM+CD44+CD47+CTCs (FIG. 2e-f).

Next, 6330 EPCAM+−CTCs of patient#1, expressing CD44 and CD47, were FACS-sorted and directly transplanted in vivo (FIG. 2g). After 8 months, bone metastasis developed, demonstrating that PICD45− EPCAM+CD44+CD47+CTCs contain functional MICs. The expression status of CD44, CD47 and c-MET was then compared in patient primary tumors, patient bone metastasis and in corresponding experimental CTC-induced bone metastases of patient#1 (after FACS sorting) and patient#2 (after hematopoietic cell-depletion). Expression of CD44 (data not shown) and CD44v6 was detected in all specimens (FIG. 2h, FIG. 7a). The CD44v6 isoform contains a small additional extracellular region known to form a highly active ternary complex with c-MET and its ligand HGF (Orian-Rousseau et al., CD44 is required for two consecutive steps in HGF/c-Met signaling. Genes Dev 16 (23), 3074-3086 (2002); Orian-Rousseau et al., CD44, a therapeutic target for metastasising tumours. Eur J Cancer 46 (7), 1271-1277 (2010)).

While only 33% of the transplanted MIC-containing CTCs expressed c-MET (FIG. 2f), c-MET expression was detected in primary tumors and bone metastases as well as in experimental CTC-induced bone metastases of both patients, suggesting that MICs also express c-MET (FIG. 2i, FIG. 7b). Strikingly, the primary tumor of patient#1, which at the time of analysis showed no signs of metastasis, did not express CD47. In contrast, CD47 expression was detected in the bone metastases of patient#1 developed seven years later, in the primary mammary tumor of metastatic patient#2 and in experimental CTC-induced bone metastases of both patients (FIG. 2j, FIG. 7c). These data indicate that CD47 expression may have been acquired at the onset of the metastatic process. Most importantly, these data show that EPCAM+CD44+CD47+CTCs contain functional MICs, which most likely also express c-MET. Since both patients belong to the luminal breast cancer subtype, we focused all further analysis on this subclass, which is the most frequent form of breast cancer. 11,12 To further address CTC heterogeneity and inter-patient CTC variation in luminal breast cancers, we analyzed EPCAM+−CTCs of four additional patients by FACS (patients #3-6, Table 1). All patients displayed a population of CD44− expressing EPCAM+−CTCs, ranging from 5%-55% (FIG. 3a). Strikingly, c-MET-expressing EPCAM+−CTCs were exclusively present within CD44+EPCAM+− CTCs (FIG. 3b, 3d). Similarly, CD47+EPCAM+−CTCs were found predominantly in the CD44+ fraction (FIG. 3c, 3e-g). Thus, CD44+CD47+c-MET+ populations could be identified in all patients, ranging from 1.4%-43.6% within EPCAM+−CTCs (FIG. 3j). It has been suggested that CTCs might down-regulate epithelial markers such as EPCAM due to epithelial to mesenchymal transition (EMT) (Polyak et al., Transitions between epithelial and mesenchymal states: acquisition of malignant and stem cell traits. Nat Rev Cancer 9 (4), 265-273 (2009)).

To address whether EMT may have occurred in patient-CTCs, an EPCAMlo gate was defined for each patient, containing half of the entire EPCAM+−CTC population (FIG. 3h). CD44+EPCAM+−CTCs were significantly enriched in EPCAMlo cells, while the majority of CD44− EPCAM+−CTCs showed higher EPCAM expression (FIG. 3h, 3i). These data are consistent with the hypothesis that partial EMT may occur during the generation of circulating MICs. The identification of c-MET expression on CTCs, on patient metastases and on CTC-induced xenografts raises the possibility that this receptor may drive metastatic disease. We therefore investigated the impact of c-MET expression on breast cancer patient outcome by tissue-microarray analysis (n=327, Table 2). The results revealed c-MET expression as an independent predictor of decreased overall-survival with a hazard ratio of 3.4 (p<0.001) (Table 4 and FIG. 8). In order to address whether this is also the case for the luminal subtype, survival analysis was also performed for HR+ patients only (n=255). Univariate-analysis showed that c-MET expression strongly correlates with decreased overall-survival (p=0.001, mean decreased by >6 years) (FIG. 4a-c, Table 2). Moreover, multivariate analysis showed that c-MET expression is an independent predictive factor for decreased overall-survival with a hazard ratio of 4.7 (p<001) (Table 3). These data demonstrate the importance of c-MET expression for luminal breast cancer patient outcome, which is correlated to metastasis occurrence and thus provide additional evidence that MICs are likely to express c-MET. In an attempt to analyze the expression pattern of CD47 and link its expression to clinical outcome in luminal patients, a cohort of 44 consecutive MO-stage HR+ tumors was examined for CD47 expression. Surprisingly, only 2/44 tumors (4.5%) were found to be CD47+, which excluded the possibility of generating statistically robust data on prognostic parameters. Strikingly, patients with the two CD47+ tumors had unusually extensive axillary lymph node involvement (11 and 36) demonstrating that the tumors had already metastasized locally. Conversely, CD47 was highly expressed on CTCs and/or metastatic tissues of all tested luminal patients (7/7) (FIG. 2i, FIG. 7c, FIG. 4d-f, Table 5). In summary, these data suggest that luminal tumor cells start expressing CD47 at the onset of the metastatic process and therefore may be useful as a diagnostic marker to stratify patients into metastatic and non-metastatic stages.

This study is the first to demonstrate that EPCAM+−CTCs within the blood of carcinoma patients contain MICs. While our results suggest that luminal breast cancers MICs can have an EPCAM+CD44+CD47+c-MET+ phenotype, the data do not exclude the possibility that other immunophenotypically-defined CTC populations may also contain MICs. The frequency of these cells was highly variable and it is likely that MICs represent a subpopulation of EPCAM+CD44+ breast CSCs (Al-Hajj et al., loc. cit.; Liu et al., Cancer stem cells from human breast tumors are involved in spontaneous metastases in orthotopic mouse models. Proc Natl Acad Sci USA 107 (42), 18115-18120 (2010)).

The typically low frequency of MICs underlines the need for improved tools to specifically distinguish MICs from CTCs. The finding that CD44v6 and c-MET are co-expressed raises the possibility that a previously predicted ternary complex together with HGF (Orian-Rousseau et al., Genes Dev, loc. cit.; Orian-Rousseau et al., Eur J Cancer, loc. cit.) may be operating in MICs. Tissue-microarray analysis suggests that c-MET expression is a major factor in the development of fatal metastasis in HR+ breast cancers, which is analogous to what has been previously reported for other tumor entities (Gherardi et al., Targeting MET in cancer: rationale and progress. Nat Rev Cancer 12 (2), 89-103 (2012)).

The finding that CD47 is specifically up-regulated at the time of metastasis formation is striking, as this may act to provide MICs with an escape mechanism from the phagocytic cells of the innate immune system during dissemination (Majeti et al., loc. cit.; Chao et al, Cancer Research, loc. cit.; Chan et al., loc. cit.; Chao et al., Cell, loc. cit.; Chao et al., Programmed cell removal: a new obstacle in the road to developing cancer. Nat Rev Cancer 12 (1), 58-67 (2011)).

The results presented here not only provide important information for the stratification of therapies to target metastasis initiation in the most frequent subtype of breast cancer, but will also support innovative approaches for more sensitive and selective methods for MIC detection and targeting in the blood of carcinoma patients.

### Methods

Detection of functional MICs within CTCs. In a cohort of 282 consecutive patients with progressive MBC, the number of CTCs was determined by the FDA-approved CellSearch® system (Veridex, Raritan, N.J., USA), as previously described. 13 From some patients, additional blood was collected in parallel in EDTA tubes and depleted using the RosetteSep® kit (catalog #15167, StemCell Technologies, Vancouver, Canada, FIG. 6). CTCs were then directly injected into the femurs of immuno-compromised mice. Metastatic xenograft model. Mice were treated according to the Swiss authorization number 1967 and the German authorization numbers G-114/08. Briefly, 2-month-old NOD/SCID/IL2ryc−/− (NSG) female mice were anesthetized by intra-peritoneal injection of 10 μL per g of a solution of 4.5 mg/kg Xylazinhydrochlorid and 90 mg/kg Ketamin. Flow cytometry-sorted or RosetteSep®-enriched CTCs were injected in the femurs of anesthetized mice, in a 20 μL solution of 1:4 10 mg/mL Matrigel (BD Biosciences, Heidelberg, Germany) in PBS (SIGMA, St Louis, Mo., USA). During narcosis, 90-day release 0.18 mg pellets of Estradiol (Innovative Research of America, Sarasota, Fla., USA) were implanted subcutaneously and renewed every 90 days during gaseous narcosis. Flow-cytometric analyses and sorting of CTCs. Patient CTCs were characterized by multi-parameter flow-cytometry (CYAN, DAKO, Eching, Germany and LSRII, BD Biosciences) or purified by flow-cytometry sorting 18 (INFLUX, BD Biosciences, Heidelberg, Germany) after depletion of hematopoietic cells using the RosetteSep® kit. The antibodies used for these experiments are the following: CD45-PB (clone H130, Biolegend, San Diego, Calif., USA), EPCAM-FITC (clone HEA-125, Miltenyi, Bergisch Gladbach, Germany), CD44-APCCy7 (clone IM7, Biolegend), CD47-PE (clone B6H12, BD Biosciences) and MET-APC (clone 95106, R&D, Minneapolis, Minn., USA). Propidium Iodide (Sigma-Aldrich, St. Louis, Mo., USA) was used to exclude dead cells. Imaging of CTC-induced metastases in mice. For in vivo imaging with volumetric computed tomography (VCT), mice were anesthetized with oxygen (0.5 L/min) and isoflurane (1-1.5 vol. %). VCT imaging was performed on a flat panel equipped volumetric computed tomograph (Volume CT, Siemens, Germany) just before sacrificing the mice (6 to 11 months after the injection of CTCs). Scan protocols were adjusted for bone (tube voltage 80 kV, tube current 50 mA, scan time 21 s, rotation speed 19 s, frames per second 120, matrix 512×512, slice thickness 0.2 cm) and liver imaging (tube voltage 80 kV, tube current 50 mA, scan time 61 s, rotation speed 10 s, frames per second 120, matrix 512×512, slice thickness 0.2 cm). No contrast agent was applied to image bone metastases. For assessment of liver metastases, mice were imaged before and 4 hours after intravenous application of liver-specific contrast agent (0.1 ml Fenestra LC, ART, Montreal, Canada). To compensate for respiratory movement of the liver, a small animal monitoring unit for motion gating was used (1025L, SA 19 Instruments, Stony Brook, N.Y.). Reconstructions of the images were done with VCT Reconstruction (kernel H95a, Afra, Germany). Imaging data were reconstructed with Osirix (Osirix Dicom Viewer, Version 3.3.1). Histology and Immunohistochemistry Mouse organs harboring metastatic growth were harvested and fixed in formalin. Bones were decalcified using an EDTA-based solvent. The tissue was subsequently embedded in paraffin wax. 2 μM thick sections were freshly cut. All tissue was stained with hematoxilin and eosin (H&E) as well as with periodic acid-Schiffs (PAS) stain. For immunohistochemistry, the following antibodies were used: CK7 (DAKO, Glostrup, Denmark, clone OV-TL 12/30, concentration 247 mg/L), human Ki67 (DAKO, clone Ki67, concentration 35 mg/L), estrogen receptor (Thermo Fisher Scientific Inc, Kalamazoo, Mich., USA, clone SP1, concentration 1:50), progesterone receptor (DAKO, clone PgR 636, concentration 53.8 mg/L), HER2 (DAKO, polyclonal rabbit, 320 mg/L), Mammaglobin (DAKO, clone 304-1A5, concentration 66.8 mg/L)), CD44v6 (R&D, Minneapolis, Minn., USA, clone 2F10, concentration 0.5 mg/mL), c-MET (Santa Cruz Technologies, Santa Cruz, Calif., USA, clone C28, concentration 0.2 mg/mL), CD47 (R&D, sheep polyclonal, concentration 0.2 mg/mL). For the detection of bound primary antibody, a DAKO Real Detection Multilink System with goat anti-mouse, anti-rabbit and anti-sheep antibodies were used, respectively. 20 Briefly, after antigen retrieval (citrate buffer, pH 6.0 in a steam pot) sections were blocked for endogenous Avidin/Biotin activity (Linaris, Dossenheim, Germany). Sections were then incubated for 30 minutes at room temperature with the primary antibody, washed and subsequently incubated with the respective secondary antibody for 20 minutes at room temperature. Sections were then incubated with horseradish peroxidase (HRP) for 20 minutes at room temperature. Sections were counterstained with hematoxylin. Isotype-matched mouse monoclonal antibodies were used as negative controls. HER2 gene copy numbers were determined by fluorescence in-situ hybridization (FISH) using the HER2/Cen17 probes from Zytomed (Berlin, Germany) as previously described (Riethdorf et al., Detection and HER2 expression of circulating tumor cells: prospective monitoring in breast cancer patients treated in the neoadjuvant GeparQuattro trial. Clin Cancer Res 16 (9), 2634-2645 (2010)).

All conventional, immunohistochemical as well as FISH stains were evaluated by two expert pathologists. Tissue-microarray analysis for c-MET expression Expression of c-MET was evaluated in a cohort of 327 patients with primary breast cancer using immunohistochemistry. All tumors were resected completely; none of the patients had distant metastasis or other malignant diseases at time of diagnosis. Corresponding clinicopathologic data were extracted from medical records and pathology reports. Grading as well as receptor status was rechecked by an experienced pathologist with a special expertise in breast pathology (CD). The distribution of clinicopathological variables in the patient cohort is shown in Table 2 first row. 96 patients died during follow up. Mean follow-up time of patients still alive (overall-survival) at the endpoint of analysis 21 was 133.4 months. A tissue-microarray including two cores with a diameter of 1.5 mm for each tumor was constructed from representative paraffin tissue samples. Expression was scored by two experienced histo-pathologists (W.W., A.St.) using a multi-headed microscope. Unclear cases were discussed until consensus was achieved. Consistent with the cancer stem cell concept, a case was scored as c-MET-positive when any of the tumor cells expressed c-MET while in those cases scored as negative, none of the cells showed any staining (Clevers, The cancer stem cell: premises, promises and challenges. Nat Med 17 (3), 313-319 (2011)).

This investigation had approval from the Charité ethics committee (project number EA1/139/05, date Jul. 28, 2008). Analysis of CD47 expression on breast cancer tissues Paraffin embedded primary MO-tumors from 97 consecutive breast cancer patients were collected from the Heidelberg Institute of Pathology and assembled into a tissue micro-array (TMA) as previously described (Macher-Goeppinger et al., Decoy receptor 3 is a prognostic factor in renal cell cancer. Neoplasia 10 (10), 1049-1056 (2008).

All tumors were resected by the Department of Gynaecology and Obstetrics, University Hospital Heidelberg, and processed in a standardized fashion after a minimum of 6 h of formalin fixation. The local ethics committee had approved the usage of all tissues employed in this study (No. 206/2005). 44 of these primary MO-tumors came from HR+ patients and were included in the analysis. CD47 expression was scored by an experienced histo-pathologist (Hans Peter Sinn, Pathologisches Institut der Universität Heidelberg, Im Neuenheimer Feld 220/221, D-69120 Heidelberg, Germany.).

One skilled in the art readily appreciates that the present invention is well adapted to carry out the objects and obtain the ends and advantages mentioned, as well as those inherent therein. The methods and reagents described herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

Those skilled in the art will recognize, or be able to ascertain using no more than routine experimentation, many equivalents to the specific embodiments of the invention described herein. Such equivalents are intended to be encompassed by the following claims. Those skilled in the art will also recognize that all combinations of embodiments, combination of aspects or features of the claims described herein are within the scope of the invention.

To the extent possible under the respective patent law, all patents, applications, publications, test methods, literature, and other materials cited herein are hereby incorporated by reference.

TABLE 1

| | | | | | CTCs/ | |
|---|---|---|---|---|---|---|
| | | | | | 7.5 mL by | |
| | | | | | CellSearch ® | |
| | | | | | at time of | |
| | | | | | flow- | |
| | | | | | cytometry | |
| PATIENT | ER | PR | HER2 | subtype | analysis | metastasis |
| #1 | 4/12 | 9/12 | 0 | Luminal A | 4000 | bone, |
| | | | | | | ventricular |
| | | | | | | wall |
| #2 | 12/12 | 12/12 | 0 | Luminal B | 600 | bone, lungs |
| #3 | 8/8 | 6/6 | 0 | Luminal A | 250 | bone, liver |
| #4 | 9/12 | 12/12 | 1+ | Luminal A | 74 | bone |
| #5 | 8/8 | 8/8 | 0 | Luminal B | 50 | bone, lungs |
| #6 | 12/12 | 3/12 | 0 | Luminal B | 59 | bone, liver |

TABLE 2
UNIVARIATE SURVIVAL ANALYSIS

| | | | | Overall | | Log-rank- |
|---|---|---|---|---|---|---|
| | | | | survival | Standard | test |
| | | Cases | Events | (months) | error | (p-value) |
| Age at | below | 156 | 35 | 234.5 | 9.4 | 0.007 |
| Diagnosis | mean | | | | | |
| | above | 171 | 61 | 189.9 | 8.6 | |
| | mean | | | | | |
| Tumor | T1 | 156 | 42 | 228.4 | 8.9 | 0.008 |
| stage | T2 | 139 | 44 | 190.5 | 10.3 | |
| | T3 | 15 | 2 | 227.9 | 30.0 | |
| | T4 | 15 | 8 | 145.4 | 28.6 | |
| Lypmh | N0 | 194 | 41 | 234.6 | 7.7 | <0.001 |
| node | N1 | 103 | 44 | 177.7 | 12.8 | |
| status | N2 | 12 | 4 | 135.1 | 34.8 | |
| | N3 | 9 | 4 | 54.4 | 7.6 | |
| Grade | G1 | 81 | 17 | 240.9 | 11.6 | 0.005 |
| | G2 | 158 | 53 | 193.9 | 10.1 | |
| | G3 | 88 | 26 | 202.3 | 13.6 | |
| Hormone | negative | 62 | 16 | 217.1 | 15.8 | 0.468 |
| receptors | positive | 255 | 78 | 206.7 | 7.7 | |
| Her2 | negative | 285 | 86 | 208.2 | 7.3 | 0.856 |
| | positive | 32 | 9 | 209.0 | 22.2 | |
| Met | negative | 69 | 11 | 253.1 | 12.3 | <0.001 |
| | positive | 256 | 85 | 193.7 | 7.8 | |
| Met (HR + | negative | 56 | 8 | 261.1 | 12.1 | <0.001 |
| cases) | positive | 199 | 70 | 185.0 | 8.5 | |

TABLE 3
MULTIVARIATE ANALYSIS OF
OVERALL SURVIVAL
(HR⁺ CASES)

| | HR | 95% Cl | p-value |
|---|---|---|---|
| Age at diagnosis | | | 0.002 |
| per year | 1.04 | 1.01-1.06 | |
| Tumor stage | | | 0.963 |
| T1 | 1.0 | | |
| T2 | 1.1 | 0.7-1.8 | |
| T3 | 0.9 | 0.2-3.9 | |
| T4 | 1.2 | 0.5-2.9 | |
| Lymph node status | | | <0.001 |
| N0 | 1.0 | | |
| N1 | 3.2 | 1.9-5.5 | |
| N2 | 2.5 | 0.7-9.0 | |
| N3 | 16.7 | 4.4-63.5 | |
| Grade | | | 0.009 |
| G1 | 1.0 | | |
| G2 | 2.7 | 1.4-5.1 | |
| G3 | 2.6 | 1.2-5.4 | |
| Met | | | <0.001 |
| negative | 1.0 | | |
| positive | 4.7 | 2.2-10.1 | |

TABLE 4
MULTIVARIATE ANALYSIS OF
OVERALL SURVIVAL
(WHOLE COHORT)

| | HR | 95% Cl | p-value |
|---|---|---|---|
| Age at diagnosis | | | <0.001 |
| per year | 1.04 | 1.02-1.07 | |
| Tumor stage | | | 0.303 |
| T1 | 1.0 | | |
| T2 | 1.5 | 0.9-2.3 | |
| T3 | 1.0 | 0.2-4.2 | |
| T4 | 1.6 | 0.7-3.8 | |
| Lymph node status | | | <0.001 |
| N0 | 1.0 | | |
| N1 | 2.4 | 1.5-3.7 | |
| N2 | 2.5 | 0.8-7.6 | |
| N3 | 19.1 | 6.1-60.5 | |
| Grade | | | 0.023 |
| G1 | 1.0 | | |
| G2 | 2.3 | 1.3-4.1 | |
| G3 | 1.7 | 0.9-3.2 | |
| Met | | | <0.001 |
| negative | 1.0 | | |
| positive | 3.4 | 1.8-6.6 | |

TABLE 5

| PATIENT | ER | PR | HER2 | subtype | CD47-positive tissue |
|---|---|---|---|---|---|
| #1 | 4/12 | 9/12 | 0 | Luminal A | Patient CTCs, patient |
| | | | | | bone metastasis and |
| | | | | | CTC-induced |
| | | | | | metastasis |
| #2 | 12/12 | 12/12 | 0 | Luminal B | Patient M1-tumor and |
| | | | | | CTC-induced metastasis |
| #3 | 8/8 | 6/8 | 0 | Luminal A | Patient CTCs |
| #4 | 9/12 | 12/12 | 1+ | Luminal A | Patient CTCs |
| #5 | 8/8 | 8/8 | 0 | Luminal B | Patient CTCs |
| #6 | 12/12 | 3/12 | 0 | Luminal B | Patient CTCs |
| #7 | 4/12 | 4/12 | 0 | Luminal | Patient ascites |

1. A method for analyzing circulating tumor cells of a patient for the presence of metastasis-initiating cells comprising the step of detecting cells exhibiting the simultaneous presence of c-MET, CD44 and CD47.

2-11. (canceled)

12. A kit for analyzing circulating tumor cells of a patient for the presence of metastasis-initiating cells comprising at least two reagents selected from:
(a) an analyte for detecting the presence of c-MET;
(b) an analyte for detecting the presence of CD44; and
(c) an analyte for detecting the presence of CD47.

13. The kit of claim 12 comprising all three reagents (a) to (c).

14. A kit for analyzing circulating tumor cells of a patient for the presence of metastasis-initiating cells comprising at least two reagents selected from:
(a) an analyte for detecting the presence of c-MET on the surface of circulating tumor cells;
(b) an analyte for detecting the presence of CD44 on the surface of circulating tumor cells; and
(c) an analyte for detecting the presence of CD47 on the surface of circulating tumor cells.

15. The kit of claim 14 comprising all three reagents (a) to (c).

16. The kit of claim 14, wherein one or more of said analytes are antibody-based analytes.

17. The kit of claim 16, wherein all three of said analytes are antibody-based analytes.

18. A method for treating a patient having circulating tumor cells exhibiting the simultaneous presence of c-MET, CD44 and CD47 comprising the step of administering one or more pharmaceutical compositions targeting at least two proteins selected from c-MET, CD44 and CD47.

19. The method of claim 18, wherein a pharmaceutical composition is administered, which comprises binding sites for at least two proteins selected from c-MET, CD44 and CD47.

20. The method of claim 19, wherein said pharmaceutical composition is a bispecific antibody or functional bispecific fragment thereof.

21. The method of claim 18, wherein said patient has breast cancer.

22. The method of claim 21, wherein said breast cancer is hormonal receptor positive breast cancer.

23. A bifunctional composition comprising targeting moieties for at least two proteins selected from c-MET, CD44 and CD47.

24. (canceled)

25. A bifunctional composition comprising targeting moieties for at least two proteins selected from c-MET, CD44 and CD47 for use in treating a patient having circulating tumor cells exhibiting the simultaneous presence of c-MET, CD44 and CD47.

26-28. (canceled)

29. A method for stratification of patients having luminal breast cancer, comprising the step of detecting the presence or absence of CD47 on primary tumors, wherein the presence of CD47 defines patients with metastatic disease, and the absence of CD47 defines patients with non-metastatic disease.

30. A method for assessing breast cancer patient outcome, comprising the step of detecting c-MET expression on primary tumors, wherein the presence of c-MET cells defines patients with lower overall-survival probability, and the absence of c-MET defines patients with higher overall-survival probability.

31. (canceled)

32. A population of human tumor cells enriched for the presence of human tumor cells exhibiting the simultaneous presence of c-MET, CD44 and CD47.

33-35. (canceled)
